(19) United States
(12) Patent Application Publication
(10) Pub. No.: US 2025/0090072 A1
(43) Pub. Date: Mar. 20, 2025

(54) SYSTEMS AND METHODS FOR UNITIZED DEVICES PLACED AT THE BEDSIDE FOR TEMPORARY RECORDING OF INTRACRANIAL EEG

(71) Applicant: Ice Neurosystems, Inc., Washington, DC (US)

(72) Inventors: Allen WAZIRI, Washington, DC (US); Ronald Emerson, New York, NY (US)

(73) Assignee: Ice Neurosystems, Inc., Washington, DC (US)

(21) Appl. No.: 18/643,487

(22) Filed: Apr. 23, 2024

Related U.S. Application Data
(63) Continuation of application No. 17/440,019, filed on Sep. 16, 2021, now abandoned.
Application No. 17/440,019 is a continuation of application No. PCT/US2020/025593, filed on Mar. 29, 2020.
(60) Provisional application No. 62/827,028, filed on Mar. 30, 2019.

(51) Int. Cl. A61B 5/293; A61B 5/00; A61B 5/01; A61B 5/026; A61B 5/03; A61B 5/145; A61B 5/305; A61B 5/31; A61B 5/372

(52) U.S. Cl. CPC A61B 5/293; A61B 5/01; A61B 5/026; A61B 5/031; A61B 5/14532; A61B 5/305; A61B 5/31; A61B 5/372; A61B 5/686; A61B 5/6868; A61B 5/7225; A61B 5/742; A61B 2562/046

(57) ABSTRACT

The invention encompasses systems and methods that allow a clinician who is untrained in the art of electroencephalography to insert and functionalize unitized intracranial electrode arrays at the bedside that, by specific design, position ground and reference electrodes in electrically “quiet” locations to record durable, high-fidelity intracortical EEG.

### CROSS-REFERENCE TO RELATED APPLICATION(S)

This application is a continuation of, and claims the benefit of priority to, U.S. application Ser. No. 17/440,019, filed on Sep. 16, 2021, which is a National Stage Application under 35 U.S.C. § 371 of International Application No. PCT/US2020/025593, filed Mar. 29, 2020, which claims the benefit of priority to U.S. Patent Application No. 62/827,028, filed on Mar. 30, 2019, all of which are incorporated herein by reference in their entireties.

### TECHNICAL FIELD

The invention encompasses systems and methods that allow a clinician who is untrained in the art of electroencephalography to insert and functionalize unitized intracranial electrode arrays at the bedside that, by specific design, position ground and reference electrodes in electrically “quiet” locations to record durable, high-fidelity intracortical EEG.

### BACKGROUND

Electroencephalography (EEG) is a technique which detects endogenous electrical activity of the brain by measuring voltage changes across pairs of recording electrodes. Such brain activity is largely generated by neurons located in the gray matter of the cerebral cortex.

EEG is exceedingly useful for monitoring brain health in patients with a variety of conditions that result in aberrations of endogenous brain electrical activity. In addition to a central role in detecting seizure activity, EEG has been shown to impart significant utility as a real-time physiological monitor in other settings where brain health may be at risk from potentially reversible causes such as decreased blood flow, decreased oxygen, or increased intracranial pressure. Such conditions are commonly seen in patients suffering from acute brain injury who are managed in an intensive care setting. Therefore, the ability to perform EEG in reliable and reproducible fashion in an emergent or intensive care setting carries great value in the clinical management of patients with acute brain injury. However, traditional EEG is difficult to perform in many clinical settings as a result of a series of technical and practical challenges.

First, traditional EEG relies on temporary fixation of metallic electrodes to the scalp of a patient. Such metallic scalp-based electrodes generally impart poor signal-to-noise characteristics due to the natural limitations of the metal-skin electrical interface, low signal amplitude of electrical potentials recorded from the scalp due to the distance from the cerebral cortex, the presence of intervening tissue (e.g. skull, skin, etc), and significant averaging effects of cortically-derived electrical signals which together limit subsequent data interpretation.

Second, application of long-term scalp arrays requires the availability of a trained technician who is skilled and experienced in the art of EEG. This trained individual is particularly important when considering the technical requirements associated with processing of raw brain-derived electrical signals into EEG signals. This processing includes initial hardware-based common-mode rejection of electrical artifact using a ground electrode and a subsequent step involving comparative signal acquisition from recording electrodes paired with a common reference electrode. The positioning and recording stability of the common reference and ground electrodes are of central and absolutely critical importance for effective EEG recording-should either provide poor signals (due to inaccurate electrical connection of wires from the ground or reference electrodes to associated amplifier hardware, incorrect positioning on the ground and reference electrodes at specific points on the scalp, etc.) or discontinuous data (due to high-impedance conditions between metal electrode and skin, complete loss of electrode contact with the skin, etc.) the entirety of an EEG recording may be rendered spurious or uninterpretable. Therefore, traditional scalp-based EEG requires that the highly trained technician place and maintain durable, high-fidelity ground and reference electrodes.

Third, following placement of traditional scalp electrodes, wires associated with each electrode are individually connected to specific inputs on hardware amplifier elements associated with the EEG recording system. Should any of the electrode wires (particularly those from the common ground or reference electrodes) be incorrectly connected at the inputs on the hardware, EEG recording will be spurious, uninterpretable or impossible.

Finally, traditional EEG software requires the user to specifically outline the “montage” of electrodes in use for a particular patient (i.e. the specific anatomic “pattern” of electrodes spaced across the head), necessitating detailed knowledge of the connections between ground, reference, and recording electrodes for accurate display of recorded EEG. Should any of the wiring connections be erroneously labelled, the entirety of an EEG recording can be interpreted in an incorrect or inappropriate fashion which carries significant clinical risk.

For all the aforementioned reasons, performance of traditional EEG for brain monitoring in patients with critical neurological injuries mandates continuous availability of a highly trained technician for initial scalp electrode placement, connection of electrode wires to associated device hardware, assignment and connection of appropriate channels for a given electrode montage in device hardware and software and subsequent maintenance of scalp electrodes.

Critically, however, in the vast majority of clinical settings in which patients with neurological injuries are treated, it is not practical or cost-effective for a trained technician to be available on a continuous basis which has severely limited the use of EEG as a continuous monitoring tool in brain injury. Therefore, devices that allow for simple, reproducible and reliable collection of high-fidelity EEG data by clinicians who are not trained in the art are therefore of great value.

Patients with severe brain injury often undergo insertion of monitoring or therapeutic devices into the skull and brain. Some such devices can detect physiological parameters such as pressure, tissue oxygen levels, rate of blood flow, etc. Other devices can serve a therapeutic function by draining cerebrospinal fluid (CSF) to relieve intracranial pressure. More recently, devices designed to directly record EEG from the brain have been used in patients with a range of brain injuries. Such devices that allow for direct brain recording have been shown to provide robust, durable and high-amplitude EEG data due to the direct contact with the “generator” of the EEG signal (e.g. neurons present within the gray matter of the cerebral cortex). Notably, such approaches using intracranial electrodes have previously used separate, independently connected scalp electrodes for reference and ground in EEG recording. However, as insertion of such devices into the brain often occurs in an emergent setting outside of a formal operating room (such as an intensive care unit or emergency room) and is performed by a clinician who is not skilled in the art of EEG, the ability to generalize this approach on a widespread clinical basis has been sorely limited.

Therefore, systems and methods that allow a clinician, untrained in the art of EEG, to position and functionalize electrode devices at the bedside for temporary recording of intracranial brain electrical activity are of significant value in the care of brain injured patients.

### SUMMARY

According to a first aspect, there is provided an intracranial electroencephalographic (EEG) device comprising a ground electrode, a reference electrode, a cortical recording array comprising at least one recording element, wherein each of the ground electrode, reference electrode and cortical recording array are fixed to a support structure, and wherein when the device is properly implanted in a subject's brain, the ground electrode and the reference electrode are positioned in a non-gray matter anatomic space and the cortical recording array is positioned to measure brain activity within a subject's gray matter brain space located in the cerebral cortex.

In one form, the cortical recording array comprises between 1-10 recording elements.

In one form, the non-gray matter anatomic space is selected from a subgaleal space, a subcortical white matter space, a space within a skull fixation device, or a cerebral ventricle space.

In one form, the reference electrode and the ground electrode are positioned in different non-gray matter anatomic spaces.

In one form, the reference electrode is in the subgaleal space and the ground electrode is in the subcortical white matter space, or the ground electrode is in the subgaleal space and the reference electrode is in the subcortical white matter space, or the reference electrode is in the subgaleal space and the ground electrode is in the ventricle space, or the ground electrode is in the subgaleal space and the reference electrode is in the ventricle space, or the reference electrode is within a space of the skull fixation device and the ground electrode is in the subcortical white matter space, or the ground electrode is within a space of the skull fixation device and the reference electrode is in the subcortical white matter space, or the reference electrode is within a space of the skull fixation device and the ground electrode is in the ventricle space, or the ground electrode is within a space of the skull fixation device and the reference electrode is in the ventricle space.

In one form, when the ground electrode is positioned in the subgaleal space, it is fixed to the support structure at a distance between 1.5 cm and 10 cm distal to the most superficial recording element of the cortical recording array.

In one form, when the ground electrode is positioned in the subgaleal space, it is fixed to the support structure at a distance of 3.5 cm distal to the most superficial recording element of the cortical recording array.

In one form, when the reference electrode is positioned in the subgaleal space, it is fixed to the support structure at a distance between 1.5 cm and 10 cm distal to the most superficial recording element of the cortical recording array.

In one form, when the reference electrode is positioned in the subgaleal space, it is fixed to the support structure at a distance of 3.0 cm distal to the most superficial recording element of the cortical recording array.

In one form, when the ground electrode is positioned in the subcortical white matter, it is fixed to the support structure at a distance between 1.0 cm and 3.0 cm proximal to the deepest recording element of the cortical recording array.

In one form, when the ground electrode is positioned in the subcortical white matter, it is fixed to the support structure at a distance of 2.0 cm proximal to the deepest recording element of the cortical recording array.

In one form, when the reference electrode is positioned in the subcortical white matter, it is fixed to the support structure at a distance between 1.0 cm and 3.0 cm proximal to the deepest recording element of the cortical recording array.

In one form, when the reference electrode is positioned in the subcortical white matter, it is fixed to the support structure at a distance of 1.5 cm proximal to the deepest recording element of the cortical recording array.

In one form, when the ground electrode is positioned in the skull fixation device, it is fixed to the support structure at a distance between 1.0 cm and 3.0 cm distal to the most superficial recording element of the cortical recording array and lies within the skull fixation device.

In one form, when the ground electrode is positioned in the skull fixation device, it is fixed to the support structure at a distance of 2.0 cm distal to the most superficial recording element of the cortical recording array and lies within the skull fixation device.

In one form, when the reference electrode is positioned in the skull fixation device, it is fixed to the support structure at a distance between 1.0 cm and 3.0 cm distal to the most superficial recording element of the cortical recording array and lies within the skull fixation device.

In one form, when the reference electrode is positioned in the skull fixation device, it is fixed to the support structure at a distance of 1.5 cm distal to the most superficial recording element of the cortical recording array and lies within the skull fixation device.

In one form, the reference electrode and/or the ground electrode make contact with a conductive element on the inner lumen of the skull fixation device that is electrically continuous with an otherwise electrically isolated conductive element in contact with the skull.

In one form, when the ground electrode is positioned in the cerebral ventricle, it is fixed to the support structure at a distance between 3.5 cm and 5.5 cm proximal to the deepest recording element of the cortical recording array.

In one form, when the ground electrode is positioned in the cerebral ventricle, it is fixed to the support structure at a distance of 5.5 cm proximal to the deepest recording element of the cortical recording array.

In one form, when the reference electrode is positioned in the cerebral ventricle, it is fixed to the support structure at a distance between 3.5 cm and 5.5 cm proximal to the deepest recording element of the cortical recording array.

In one form, when the reference electrode is positioned in the cerebral ventricle, it is fixed to the support structure at a distance of 4.0 cm proximal to the deepest recording element of the cortical recording array.

In one form, the device further comprises a ventricular cerebrospinal fluid drainage function.

In one form, the cortical recording array is positioned within or adjacent to the gray matter brain space of the cerebral cortex.

In one form, the device further comprises physiological sensors capable of measuring intracranial pressure, oxygen concentration, glucose level, blood flow or tissue perfusion, tissue temperature, electrolyte concentration, tissue osmolarity, a parameter relevant to brain function and/or health, or any combination thereof.

In one form, the ground electrode, the reference electrode, and/or the recording element is made of metal, an organic compound or other electrically conductive material.

In one form, the support structure is made of plastic or a biocompatible material.

In one form, the support structure is flexible or rigid.

In one form, the recording element, the reference electrode, and the ground electrode are circumferentially arranged around the support structure.

In one form, the support structure is cylindrical.

In one form, the recording element, the reference electrode, and/or the ground electrode is between 0.5 mm and 4.0 mm in width.

In one form, the device further comprises an interface connected to a processor capable of processing brain activity.

In one form, brain activity is measured by at least one parameter selected from:
(a) average voltage level;
(b) root mean square (rms) voltage level and/or a peak voltage level;
(c) derivatives involving fast Fourier transform (FFT) of recorded brain activity, including spectrogram, spectral edge, peak values, phase spectrogram, power, or power ratio; also including variations of calculated power such as average power level, rms power level and/or a peak power level;
(d) measures derived from spectral analysis such as power spectrum analysis; bispectrum analysis; density; coherence; signal correlation and convolution;
(e) measures derived from signal modeling such as linear predictive modeling or autogressive modeling;
(f) integrated amplitude;
(g) peak envelope or amplitude peak envelope;
(h) periodic evolution;
(i) suppression ratio;
(j) coherence and phase delays;
(k) wavelet transform of recorded electrical signals, including spectrogram, spectral edge, peak values, phase spectrogram, power, or power ratio of measured brain activity;
(l) wavelet atoms;
(m) bispectrum, autocorrelation, cross bispectrum or cross correlation analysis;
(n) data derived from a neural network, a recursive neural network or deep learning techniques;
or (o) identification of the recording element(s) detecting local minimum or maximum of parameters derived from (a-n).

In one form, the brain activity is measured by categorical measures of values selected from volts (V), hertz (Hz), and/or or derivatives and/or ratios thereof.

In one form, the processor is capable of processing, filtering, amplifying, digitally transforming, comparing, storing, compressing, displaying, and/or otherwise transmitting the brain activity detected by the cortical recording array.

In one form, the processor comprises hardware and/or software that analyzes, manipulates, displays, correlates, stores and/or otherwise transmits brain electrical activity.

In one form, the processor identifies the ground electrode, the reference electrode and the cortical recording array in automated fashion for a selected electrode configuration.

In one form, the processor uses the ground electrode selected in automated fashion to perform common-mode rejection for EEG signals recorded by a selected electrode configuration.

In one form, the processor uses the reference electrode selected in automated fashion to generate referential EEG recordings based on brain electrical signals detected by the cortical recording array.

In one form, the processor may further perform mathematical derivation of referential EEG recordings from individual recording elements of the cortical recording array to generate synthetic EEG data channels.

In one form, the device, the interface, and the processor are integrated with one another, the processor and the interface are integrated with one another, or the device and the interface are integrated with one another.

In one form, the interface is a physical interface.

In one form, the interface is a wireless interface.

In one form, the interface is implanted within the patient.

In one form, the interface is capable of filtering, amplifying, digitally transforming, compressing and/or transmitting brain activity detected by the cortical recording array.

### BRIEF DESCRIPTION OF DRA WINGS

Embodiments of the present invention will be discussed with reference to the accompanying drawings wherein:

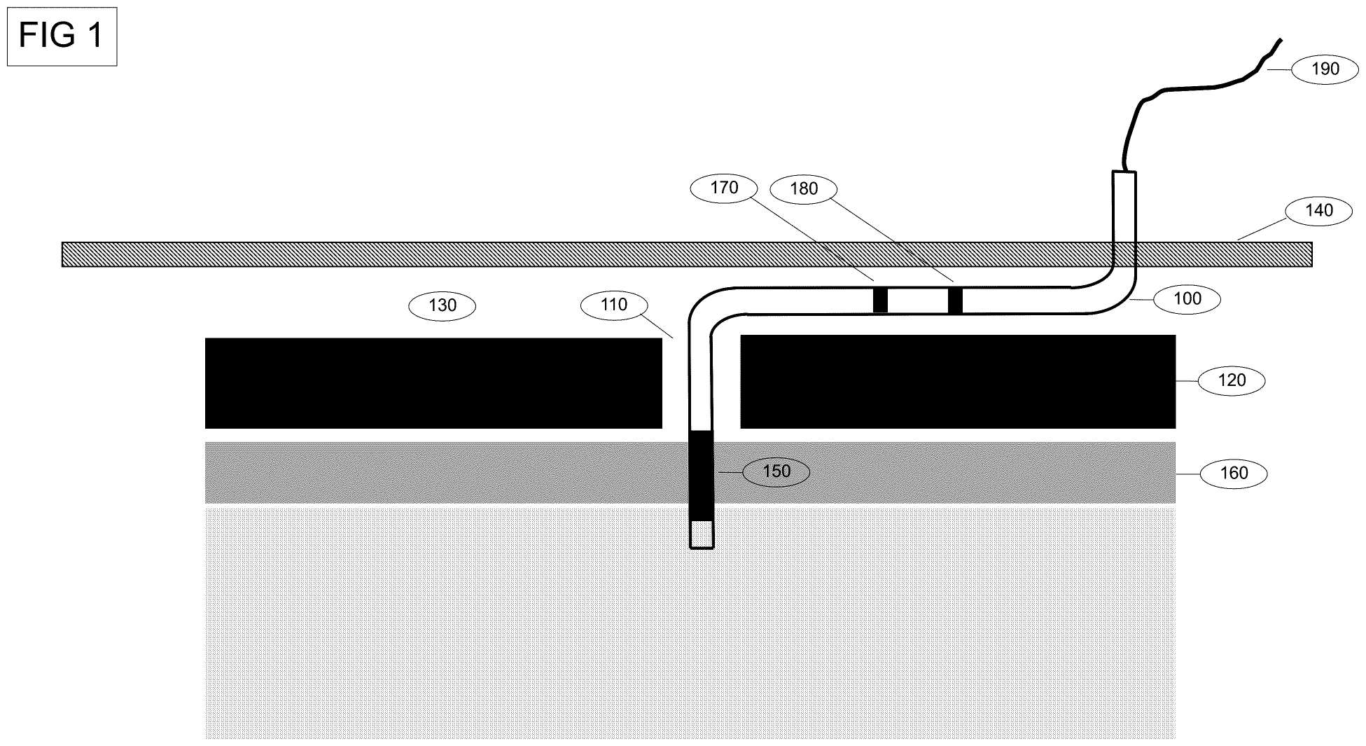

FIG. 1 depicts an intracranial EEG device according to a first aspect;

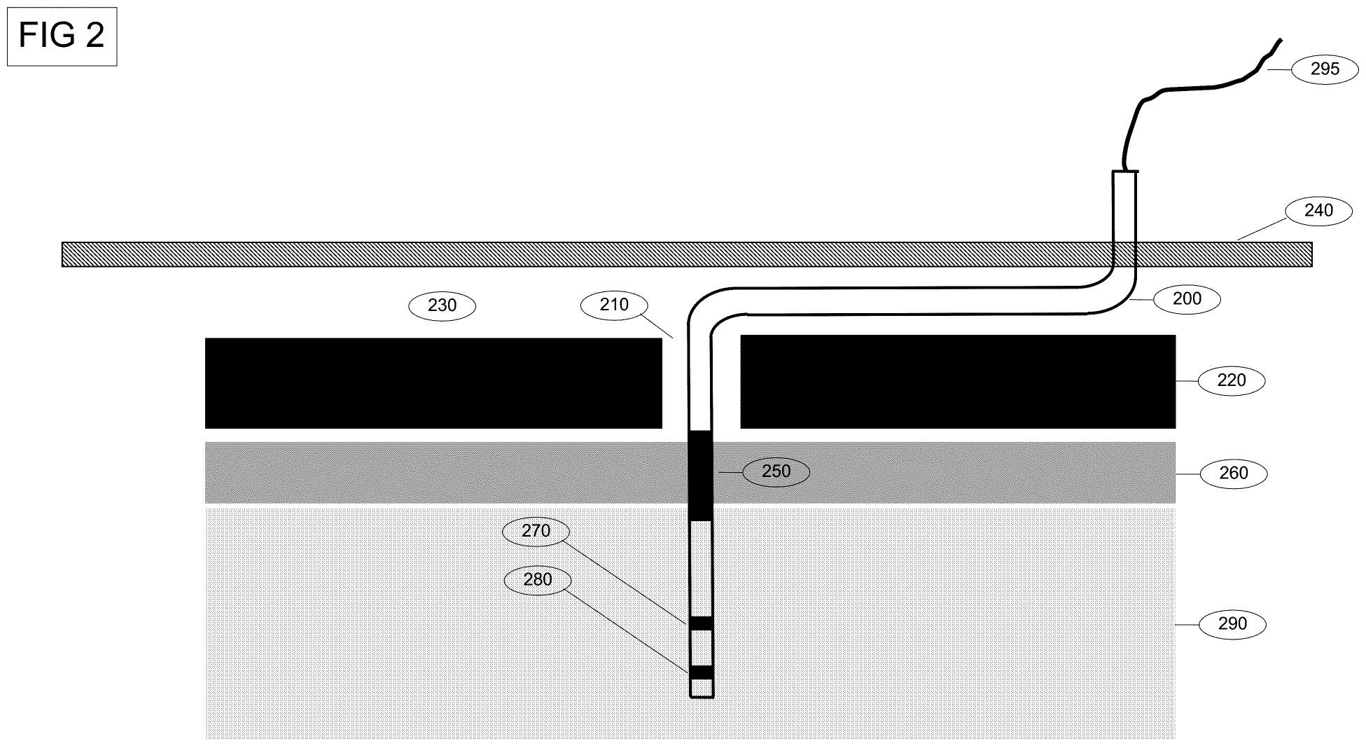

FIG. 2 depicts an intracranial EEG device according to a second aspect;

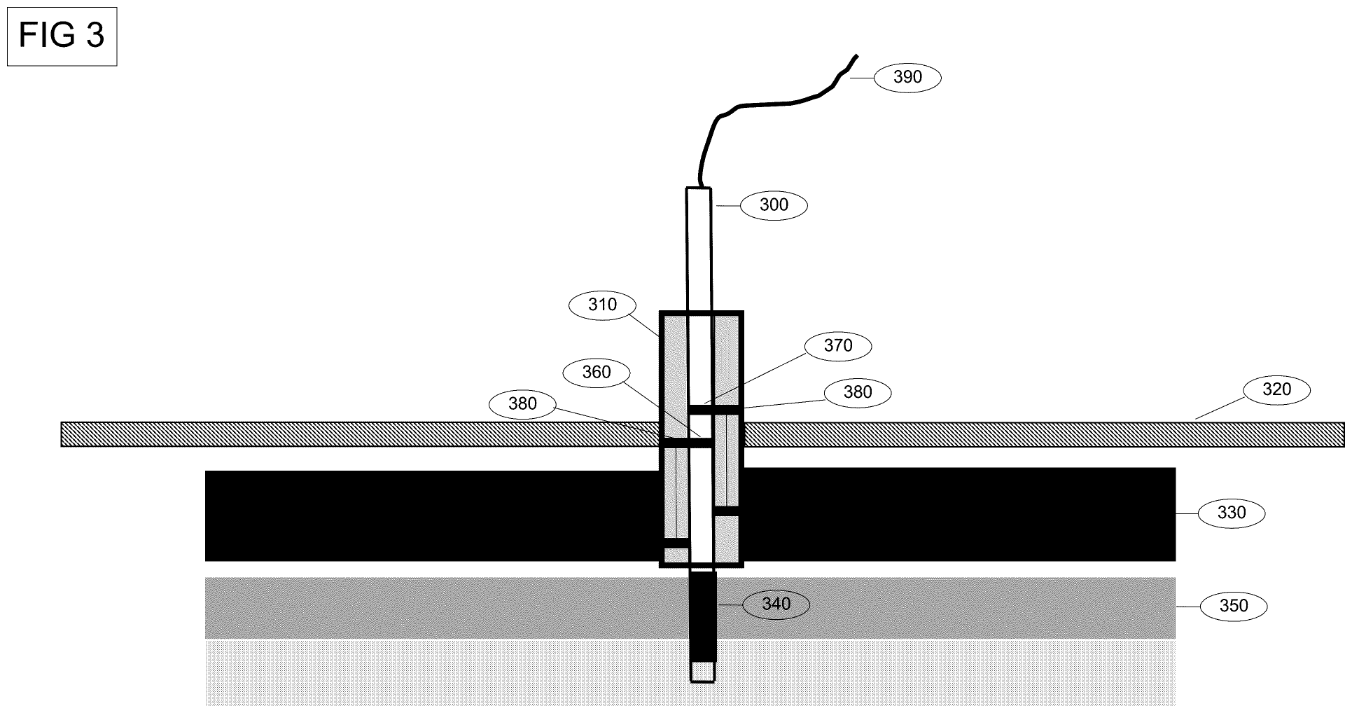

FIG. 3 depicts an intracranial EEG device according to a third aspect;

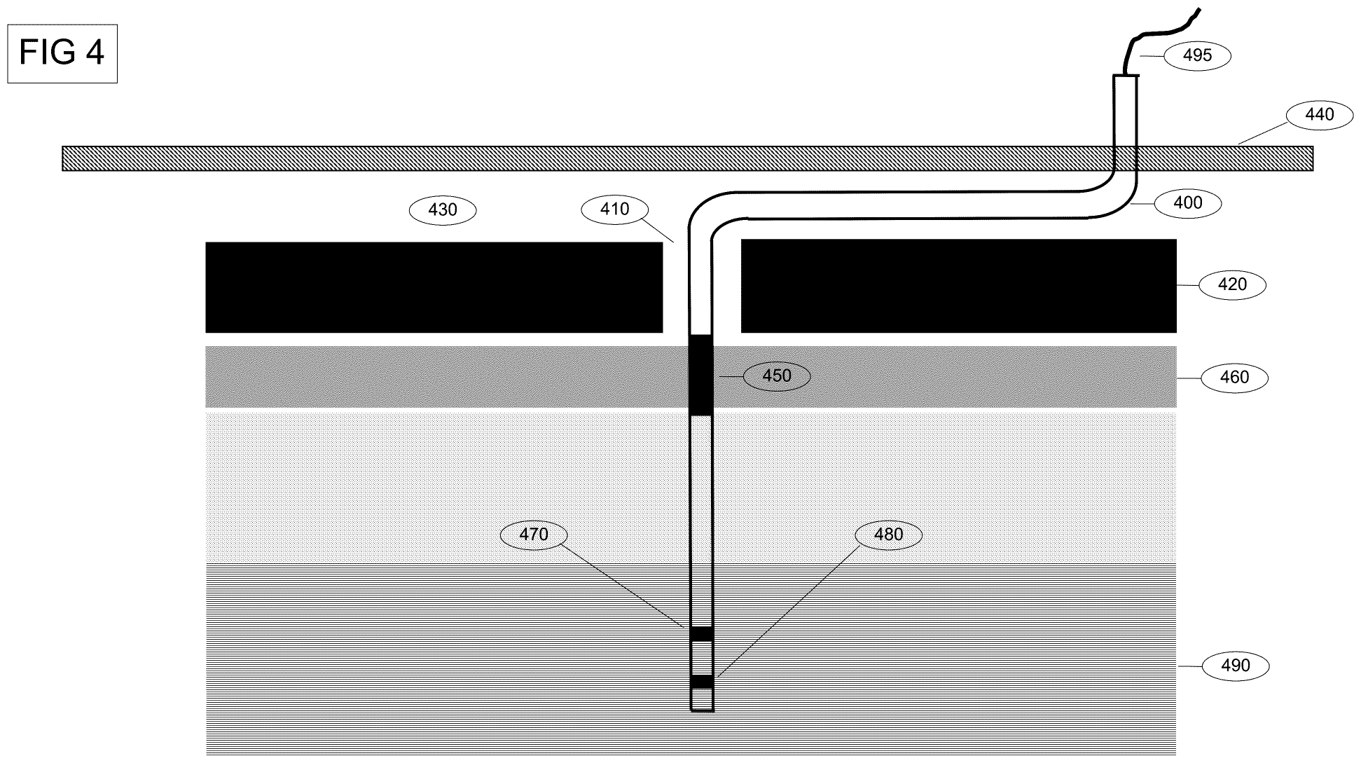

FIG. 4 depicts an intracranial EEG device according to a fourth aspect;

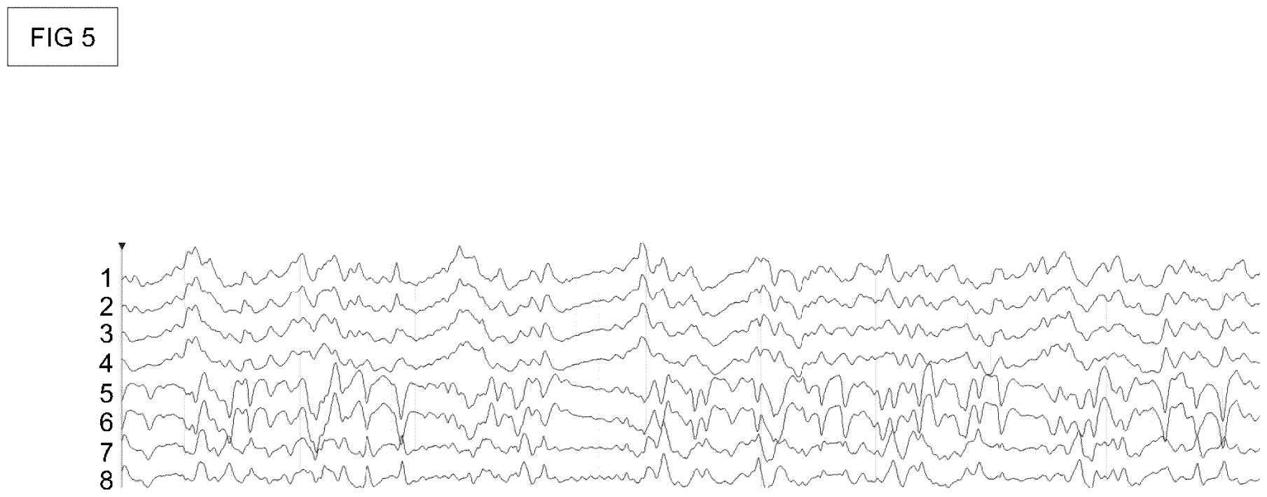

FIGS. 5 to 7 provide representative EEG data generated in an anesthetized porcine model using a series of electrode arrays with known intercontact spacing;

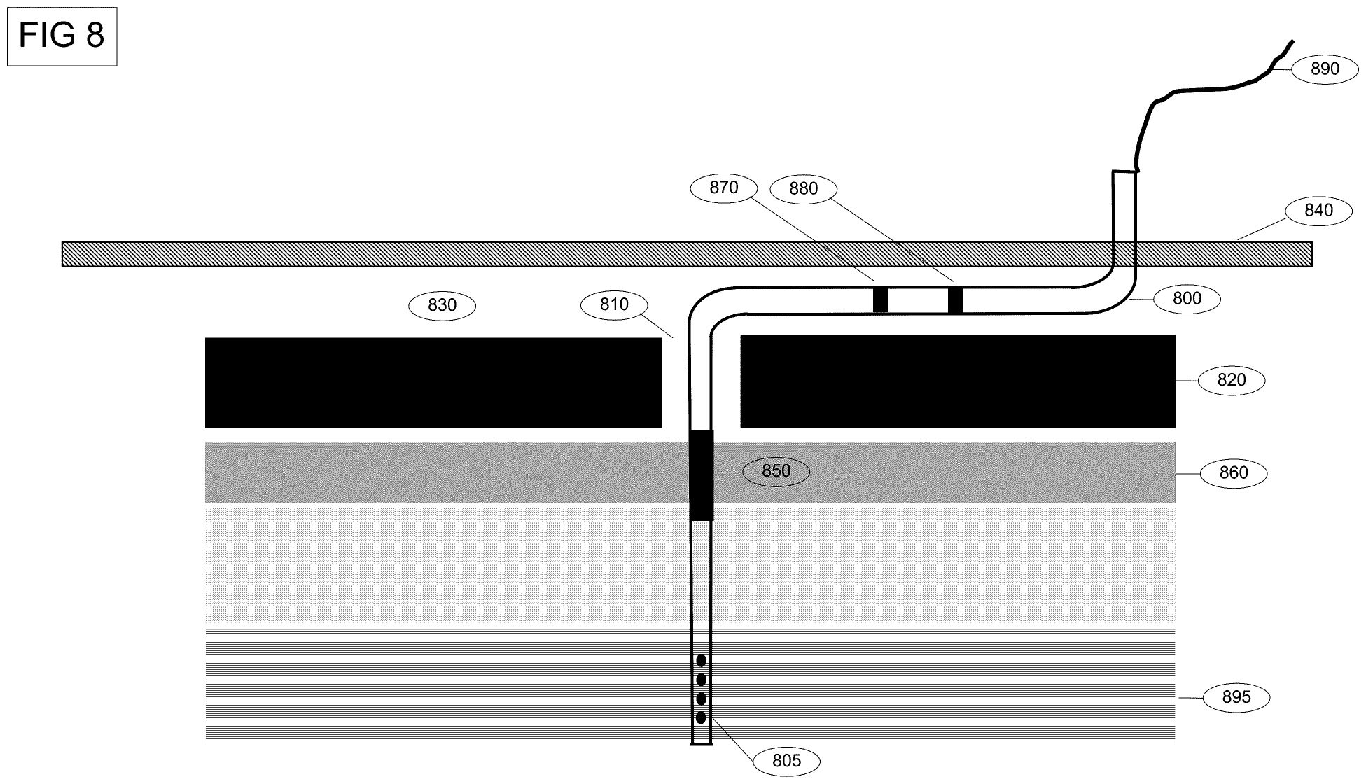

FIG. 8 depicts an intracranial EEG device according to a fifth aspect; and

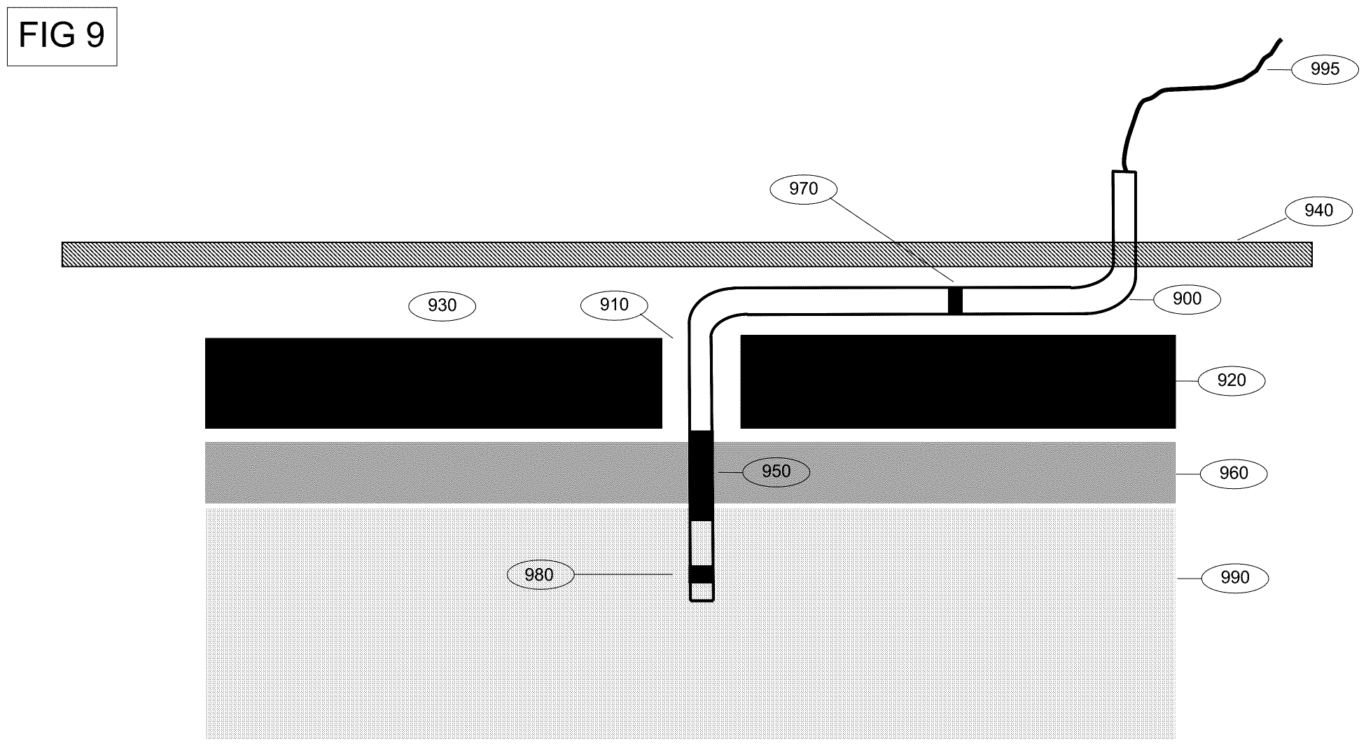

FIG. 9 depicts an intracranial EEG device according to a sixth aspect.

### DESCRIPTION OF EMBODIMENTS

As used herein, a “reference electrode” refers to a contact (preferably also made of metal) designed to act as a common member of variable electrode pairs as a control allowing for the comparison of brain activity detected by one or more recording elements on the implantable array. For example, the reference electrode can allow for comparison of brain activity detected by multiple recording elements.

As used herein, a “ground electrode” refers to a recording element which serves to provide information about globally recorded electrical signals that derive from non-physiological sources (such as local electrical equipment) and therefore allow for common-mode rejection of such non-physiological signals.

As used herein, a “recording element” is a contact which is capable of detecting brain electrical activity.

As used herein, a “subgaleal space” refers to the anatomic compartment of the scalp which lies below the epidermis and galea aponeurosis (the fascial layer of the scalp) and the periosteum and bone of the skull. The subgaleal space is a naturally occurring, avascular region that can be easily accessed and traversed using specialized tools without risk of significant injury, bleeding, risk of intracranial infection, or other major medical complication.

As used herein, a “subcortical white matter space” refers to white matter of the brain that is located within the cerebral hemispheres deep to the gray matter of the cerebral cortex.

As used herein, a “skull fixation device” refers to a hardware element that is designed to be implanted within or otherwise secured to the skull that allows for passage and stabilization of a separate hardware element (e.g. an electrode array) through an opening in the skull.

As used herein, a “cerebral ventricle space” refers to an anatomic position within one of the cerebrospinal fluid-containing chambers within the brain.

As used herein, a “support structure” refers to a structure (a) capable of housing the reference, the ground and the recording elements; (b) capable of transmitting the electrical signal generated by the brain to the associated processor; and (c) capable of being inserted through the skin, optionally tunneled through the subgaleal space, through a burr hole in the subject's skull and with at least a portion maintained intracranially. The support structure may be designed for passage through a separate piece of equipment that is tunneled through the subgaleal space and/or skull or the support structure itself may contain the necessary elements to allow for independent passage.

As used herein, a “circumferential arrangement” is defined as fully wrapping around the support structure so that geographically specific electrical signals (for example those originating only on one side of the array) can be recorded no matter the rotational position of the array in relation to the electrical signal. This therefore allows for pandirectional recordings with optimal tissue contact and/or eliminates need for a specific orientation of the device.

As used herein, “proximal” and “distal” are used to denote positions along the support structure, with the most proximal aspect of the device residing at the tip of the device (e.g. deepest point of insertion) within the brain and the most distal aspect of the device residing at the farthest point (e.g. the end of the device not inserted in the brain) from the tip of the device inserted in the brain.

As used herein, “deep” and “shallow” are used to describe position of a device relative to the brain surface. For example, “deeper” insertion denotes a position along the structure of a device that is inserted farther into the substance of the brain, while “superficial” means a position along the structure of a device that is farther from the tip of the device inserted within the brain.

Referring to FIGS. 1 to 4, there are shown four intracranial electroencephalographic (EEG) devices 100, 200, 300, 400 for implanting in a subject's brain. Each device includes a ground element, a reference element and a cortical recording array comprising at least one recording element, where each of the elements are fixed to a support structure.

When the device is properly implanted in the subject's brain, the ground element and the reference element are positioned in a non-gray matter anatomic space and the cortical recording array is positioned to measure brain activity within the subject's gray matter brain space located in the cerebral cortex.

The cortical recording array may comprise between 1 and 10 recording elements, which are organized and positioned at specific points along the length of the support structure to be placed within or in contact with the cerebral cortex to detect high-amplitude brain electrical activity.

The ground and reference elements are placed at specific distances from the recording elements along the support structure that, based on measured characteristics of human brain and cranial anatomy, results in positioning of the ground and reference elements in non-gray matter, low-amplitude tissue compartments sometimes referred to as “quiet” regions. As will be described in further detail below, these locations may be selected from a subgaleal space, a subcortical white matter space, a space within a skull fixation device, or a cerebral ventricle space.

The device is connected either with a wire or wirelessly to a hardware interface component that is preconfigured for known inputs from a specified electrode array. The hardware interface component is connected to a processor which allows a clinician to select a particular element configuration, whereby the processor then identifies the ground and reference elements in automated fashion for that particular device configuration.

Referring now to FIG. 1, where there is shown an intracranial EEG device 100 according to a first aspect. The device 100 is designed for placement through a burr hole 110 in the skull 120 and tunnelled through the subgaleal space 130 and scalp 140 at a distance from the insertion site. The cortical recording array 150 is located within the gray matter of the cerebral cortex 160 and the ground 170 and reference 180 electrodes are positioned to reside in the subgaleal tissue compartment 130 (also referred to as the subgaleal space). Ground, reference and recording electrodes are connected by a wire 190 to a hardware interface component.

The ground electrode 170 may be fixed to the support structure between 1.5 cm and 10 cm (and ideally 3.5 cm) distal to the most superficial recording element of the cortical recording array 150. The reference electrode 180 may be fixed to the support structure between 1.5 cm and 10 cm (and ideally 3.0 cm) distal to the most superficial recording element of the cortical recording array 150.

In regard to range of positioning for ground or reference electrodes within the subgaleal space (1.5 to 10.0 cm distal to the most superficial contact in the cortical recording array), several anatomical measurements and practical applications specific to the claimed device were considered. Through clinical experience and measurements performed by the inventors, the variation of human skull thickness in the region of device insertion ranges from 1.0 cm to 2.0 cm. Through consideration of device design and surgical procedures associated with device insertion, devices may be tunneled through the subgaleal space and brought out through the skin at distances that range from 0.5 cm at minimum to 8.0 cm at a maximum from the opening in the skull used for device insertion. Therefore, in the case of the most “proximally” oriented location for the reference or ground contact (i.e. 1.5 cm), it was assumed that there was minimum 1.0 cm of skull thickness and 0.5 cm distance from the opening in the skull to the contact within the subgaleal space. In the case of the most “distally” oriented location for the reference or ground contact (i.e. 10.0 cm), it was assumed that there was maximum skull thickness of 2.0 cm and 8.0 cm distance from the opening in the skull to the contact located within the subgaleal space.

Referring now to FIG. 2, where there is shown an intracranial EEG device 200 according to a second aspect. The device 200 is designed for placement through a burr hole 210 in the skull 220 and tunnelled out through the subgaleal space 230 and scalp 240 at a distance to the insertion site. The cortical recording array 250 is located within the gray matter of the cerebral cortex 260 and the ground 270 and reference 280 electrodes are positioned to reside in the subcortical white matter compartment 290. Ground, reference and recording electrodes are connected by a wire 295 to a hardware interface component.

In this example, the ground electrode 270 may be fixed to the support structure between 1 cm and 3 cm (and ideally 2 cm) proximal to the deepest recording element of the cortical recording array 250. The reference electrode 280 may be fixed to the support structure between 1 cm and 3 cm (and ideally 1.5 cm) proximal to the deepest recording element of the cortical recording array 250. The relative orientation of the reference electrode 280 vs the ground electrode 270 are not dependent upon one another, but rather dependent upon the deepest recording element.

In regard to range of positioning for ground or reference electrodes within the subcortical white matter compartment (1.0 to 3.0 cm proximal to the deepest contact on the cortical recording array), several anatomical measurements for optimized device function and specific to the claimed device were considered. Through clinical experience and measurements performed by the inventors the boundaries of the subcortical white matter reliably begin approximately 1.0 cm below the deep border of the gray matter of the cerebral cortex. The deep boundary of the subcortical white matter lies at approximately 3.0 cm from the deep border of the gray matter of the cerebral cortex and is limited by the CSF-containing lateral ventricle, as in some cases lateral ventricle can lie within 3.5 cm from the cortical surface. Therefore, the most superficial white matter location for the reference or ground contacts within the subcortical white matter can lie at 1.0 cm from the deep border of the gray matter of the cerebral cortex and the deepest white matter location for the reference or ground electrode can lie at 3.0 cm from the deep border of the gray matter of the cerebral cortex.

Referring now to FIG. 3, where there is shown an intracranial EEG device 300 according to a third aspect. The device 300 is designed for placement through a skull fixation device 310 which passed through an opening in the skin 320 and is placed through a hole in and is in direct contact with the bone of the skull 330. The cortical recording array 340 is located within the gray matter space of the cerebral cortex 350 and the ground 360 and reference 370 electrodes are positioned within the skull fixation device 310. The ground 360 and reference 370 electrodes make contact with electrically-isolated independent conductive elements 380 that make external independent electrical contact with the skull 330. Ground, reference and recording electrodes are connected by a wire 390 to a hardware interface component.

In this configuration, the ground electrode 360 may be fixed to the support structure between 1 cm and 3 cm (and ideally 2.0 cm) distal to the most superficial recording element of the cortical recording array 340. The reference electrode 370 may be fixed to the support structure between 1 cm and 3 cm (and ideally 1.5 cm) distal to the most superficial recording element of the cortical recording array 340. Again, the relative orientation of the reference electrode 370 and the ground electrode 360 is not dependent upon one another, but rather dependent upon the most superficial recording element.

In regard to range of positioning for ground or reference electrodes within a skull fixation device (1.0 to 3.0 cm distal to the most superficial contact on the cortical recording array), several anatomical measurements and engineering aspects specific to the claimed device were considered. As above, the thickness of the human skull in the region of device insertion ranges from 1.0 cm to 2.0 cm. In addition, typical height of the skull fixation device outside the associated opening of the skull ranges from 1.0 to 3.0 cm. Given requirements for the proposed device that would create electrical contacts on the inner lumen of the fixation device that would interface with reference or ground elements along the support structure of the claimed device, as well as provided necessary distance from the opening of the skull fixation device on either side of the interface points, the minimum distance distal to the most superficial contact on the cortical recording array for the reference or ground contact would be 1.0 cm and the maximum distance distal to the most superficial contact on the recording array for the reference or ground contact would be 3.0 cm.

Referring now to FIG. 4, where there is shown an intracranial EEG device 400 according to a fourth aspect. The device 400 is designed for placement through a burr hole 410 in the skull 420 and tunnelled out through the subgaleal space 430 and scalp 440 at a distance from the insertion site. The cortical recording array 450 is located within the gray matter of the cerebral cortex 460 and the ground 470 and reference 480 electrodes are positioned to reside in a cerebral ventricle compartment 490. Ground, reference and recording electrodes are connected by a wire 495 to a hardware interface component.

In this embodiment, the ground electrode 470 may be fixed to the support structure between 3.5 cm and 5.5 cm (and ideally 5.5 cm) proximal to the deepest recording element of the cortical recording array 450. The reference electrode 480 may be fixed to the support structure between 3.5 cm and 5.5 cm (and ideally 4.0 cm) proximal to the deepest recording element of the cortical recording array 450. As in the other embodiments, the relative position of the reference and ground electrodes are dependent upon the position of the deepest recording element.

Referring now to FIG. 8, where there is shown an intracranial EEG device 800 with combined cerebrospinal fluid (CSF) drainage function 805 according to a fifth aspect. The device 800 is hollow with a central lumen designed for placement through a burr hole 810 in the skull 820 and tunnelled out through the subgaleal space 830 and scalp 840 at a distance from the insertion site. The cortical recording array 850 is located within the gray matter of the cerebral cortex 860 and the reference 870 and ground 880 electrodes are positioned to reside in in the subgaleal space 830. Ground, reference and recording electrodes are connected by a wire 890 to an external hardware interface.

In this embodiment, the ground electrode 880 may be fixed to the support structure between 1.5 cm and 10 cm (and ideally 3.5 cm) distal to the most superficial recording element of the cortical recording array 850. The reference electrode 870 may be fixed to the support structure between 1.5 cm and 10 cm (and ideally 3 cm) distal to the most superficial recording element of the cortical recording array 150. The CSF drainage function 805 consisting of holes within the support structure to drain through the hollow lumen of the device to an external collection system is located at the deepest aspect of the support structure within the cerebral ventricle 895.

In regard to range of positioning for ground and reference electrodes within the CSF-containing lateral ventricle (3.5 to 5.5 cm distal to the deepest contact on the cortical recording array), several anatomical measurements for optimized device function and design elements specific to the claimed device were considered. Through clinical experience and measurements performed by the inventors the boundary of the CSF-containing lateral ventricle ranges from a minimum of 3.0 cm to a maximum of 4.0 cm with an average of 3.5 cm from the deepest border of the gray matter of the cerebral cortex. The size of the lateral ventricle within which intraventricular portion of the device would lie ranges from 1.5 to 2.5 cm. As such, the range within which the reference or ground contacts can be positioned proximal to the cortical recording array along the support structure would be 3.5-5.5 cm with an ideal iteration harboring a reference contact at 4.0 cm proximal to the cortical recording array and the ground electrode at 5.5 cm proximal to the cortical recording array.

The positions of the recording elements, the ground electrode and the reference electrode on the intracranial EEG device were determined by the inventors after placing more than 50 individual electrodes in human patients and confirmed using correlative experiments in a porcine model. Considerations that were taken into account when determining the optimal positions of the sensors on the intracranial EEG device include brain anatomy, observed differences in patient to patient variances, and the type of data desired to be obtained from the intracranial EEG device.

It will be appreciated that any one of the above embodiments may further comprise physiological sensors capable of measuring parameters such as intracranial pressure, oxygen concentration, glucose level, blood flow, tissue perfusion, tissue temperature, electrolyte concentration, tissue osmolarity, or any other parameter relevant to brain function and/or health.

According to a further aspect, there may be an intracranial EEG device where the reference electrode and the ground electrode are positioned in different non-gray matter anatomic spaces, with the following configurations being possible:
a. the reference electrode is in the subgaleal space and the ground electrode is in the subcortical white matter space; or
b. the ground electrode is in the subgaleal space and the reference electrode is in the subcortical white matter space; or
c. the reference electrode is in the subgaleal space and the ground electrode is in the ventricle space; or
d. the ground electrode is in the subgaleal space and the reference electrode is in the ventricle space; or
e. the reference electrode is within a space of the skull fixation device and the ground electrode is in the subcortical white matter space; or
f. the ground electrode is within a space of the skull fixation device and the reference electrode is in the subcortical white matter space; or
g. the reference electrode is within a space of the skull fixation device and the ground electrode is in the ventricle space; or
h. the ground electrode is within a space of the skull fixation device and the reference electrode is in the ventricle space.

Referring now to FIG. 9, where there is shown an intracranial EEG device 900 according to a sixth aspect wherein ground and reference electrodes are located in different compartments. The device 900 is designed for placement through a burr hole 910 in the skull 920 and tunnelled through the subgaleal space 930 and scalp 940 at a distance from the insertion site. The cortical recording array 950 is located within the gray matter of the cerebral cortex 960 and the ground 970 and reference 980 electrodes are positioned to reside in the subgaleal tissue compartment 930 and white matter compartment 990 respectively. Ground, reference and recording electrodes are connected by a wire 995 to a hardware interface component.

The ground electrode 970 may be fixed to the support structure between 1.5 cm and 10 cm (and ideally 3.5 cm) distal to the most superficial recording element of the cortical recording array 950. The reference electrode 980 may be fixed to the support structure between 1.0 cm and 3.0 cm (and ideally 1.5 cm) proximal to the cortical recording array 950 to lie within the white matter compartment 990.

While the devices shown in the embodiments above features a cortical recording array positioned within the gray matter space of the cerebral cortex, it will be appreciated that the cortical recording array may also be positioned in immediate contact with the gray matter surface of the cerebral cortex, as may be performed with subdural electrode arrays.

It will be appreciated that the ground electrode, the reference electrode and the or each recording element may be made from a metal, an organic compound or any other suitable electrically conductive material.

The support structure may be made from a plastic or other suitable biocompatible material. The support structure may either be flexible or rigid, and may have a generally cylindrical form.

The or each recording element, reference electrode and ground electrode may be circumferentially formed around the support structure and may be between 0.5 mm and 4 mm in width.

All of the above described devices will feature an interface for connection to a processor capable of processing brain activity, which may be measured by categorical measure of values selected from volts (V), hertz (Hz), and/or derivatives and/or ratios thereof, wherein brain activity is measured by at least one parameter selected from:
a. average voltage level;
b. root mean square (rms) voltage level and/or a peak voltage level;
c. derivatives involving fast Fourier transform (FFT) of recorded brain activity, including spectrogram, spectral edge, peak values, phase spectrogram, power, or power ratio; also including variations of calculated power such as average power level, rms power level and/or a peak power level;
d. measures derived from spectral analysis such as power spectrum analysis; bispectrum analysis; density; coherence; signal correlation and convolution;
e. measures derived from signal modeling such as linear predictive modeling or autogressive modeling;
f. integrated amplitude;
g. peak envelope or amplitude peak envelope;
h. periodic evolution;
i. suppression ratio;
j. coherence and phase delays;
k. wavelet transform of recorded electrical signals, including spectrogram, spectral edge, peak values, phase spectrogram, power, or power ratio of measured brain activity;
l. wavelet atoms;
m. bispectrum, autocorrelation, cross bispectrum or cross correlation analysis;
n. data derived from a neural network, a recursive neural network or deep learning techniques; or
o. identification of the recording element(s) detecting local minimum or maximum of parameters derived from (a-n).

The processor may be capable of processing, filtering, amplifying, digitally transforming, comparing, storing, compressing, displaying, and/or otherwise transmitting the brain activity detected by the cortical recording array. The processor may comprise hardware and/or software that analyzes, manipulates, displays, correlates, stores and/or otherwise transmits brain electrical activity. The processor may identify the ground electrode, the reference electrode and the cortical recording array in automated fashion for a selected electrode configuration. The processor may use the ground electrode selected in automated fashion to perform common-mode rejection for EEG signals recorded by a selected electrode configuration. The processor may use the reference electrode selected in automated fashion to generate referential EEG recordings based on brain electrical signals detected by the cortical recording array. The processor may further perform mathematical derivation of referential EEG recordings from individual recording elements of the cortical recording array to generate synthetic EEG data channels.

In one form the device, the interface and the processor may be integrated with one another. In another form the processor and the interface may be integrated with one another. In another form, the device and the interface may be integrated with one another. In one form, the interface may be a physical interface, in another form it may be a wireless interface. In one form the interface may be implanted within the subject. In one form the interface may be capable of filtering, amplifying, digitally transforming, compressing and/or transmitting brain activity detected by the cortical recording array.

Referring now to FIGS. 5 to 7, which provide representative EEG data generated in an anesthetized porcine model using a series of electrode arrays with known intercontact spacing. Following induction of general anesthesia, a burr hole was created in the right frontal region and a recording electrode array (1.12 mm contacts, 2.2 mm intercontact spacing) was placed under direct vision into the brain until the last contact was just below the cortical surface. Of note, measured variability in the porcine and human skull thickness in this region ranges from 1.0 cm to 2.0 cm. Measured variability in the distance from the cortical surface to the inner surface of the skull ranges from 0.5 cm to 0.1 cm in both the porcine and human settings. Measured variability in the thickness of the cortical gray matter range from roughly 2.5 mm to 5.0 mm in both the porcine and human systems.

In the experiment providing representative data for FIG. 5, following placement of the recording electrode array the distance from the cortical surface to the inner surface of the brain was measured at 0.5 mm and the skull thickness was 1.5 cm. Following insertion of the recording array, a separate electrode array (the reference/ground array, with intercontact spacing of 5 mm) was tunneled laterally from the burr hole through the subgaleal space. This approach brought the first contact (assigned as the reference electrode) on the reference/ground array 1.0 cm from the burr hole, resulting in a total distance from the cortical surface of 3.0 cm and brought the second contact (assigned as the ground electrode) 3.5 cm from the cortical surface. Time on the x-axis is measured in seconds per division. Contacts labelled on the y-axis range from 1 (deepest) to 8 (shallowest) within the brain.

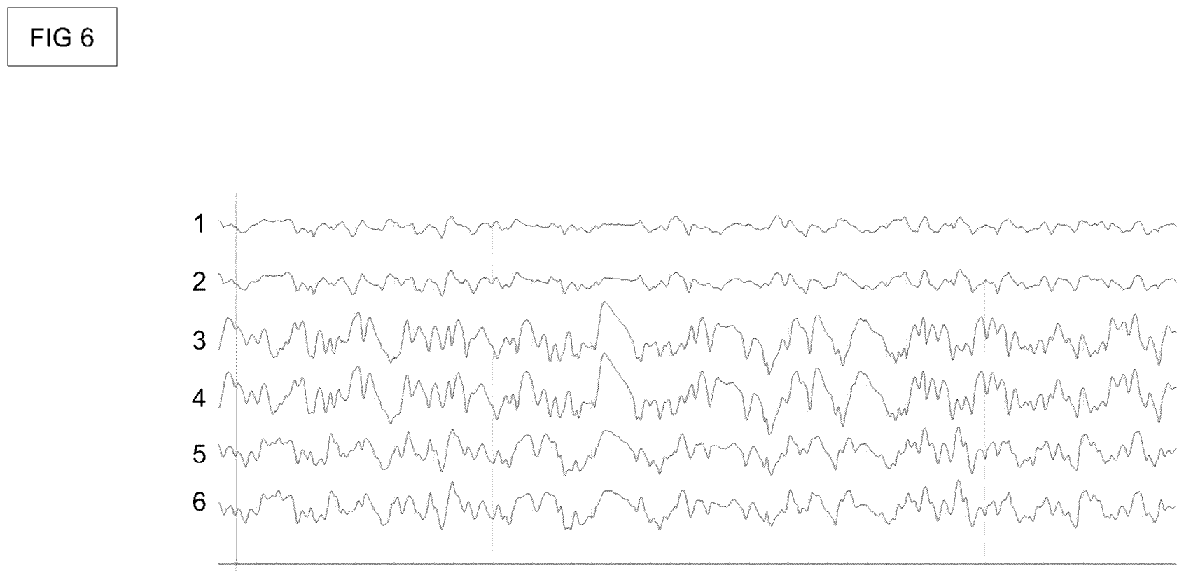

In the experiment providing representative data for FIG. 6, following placement of the cortical recording array a separate reference/ground electrode array (again with 5 mm intercontact spacing) was placed through a separate cortical approach adjacent to the recording electrode array. Using observed cortical thickness in the animal providing representative data, the deepest contact on the reference/ground array (assigned as the reference electrode) was located at 2.0 cm from the closest contact on the recording array, and the next deepest contact on the reference/ground array (assigned as the ground electrode) was located at 1.5 cm from the closest contact on the recording array. Time on the x-axis is measured in seconds per division. Contacts labelled on the y-axis range from 1 (deepest) to 6 (shallowest) within the brain.

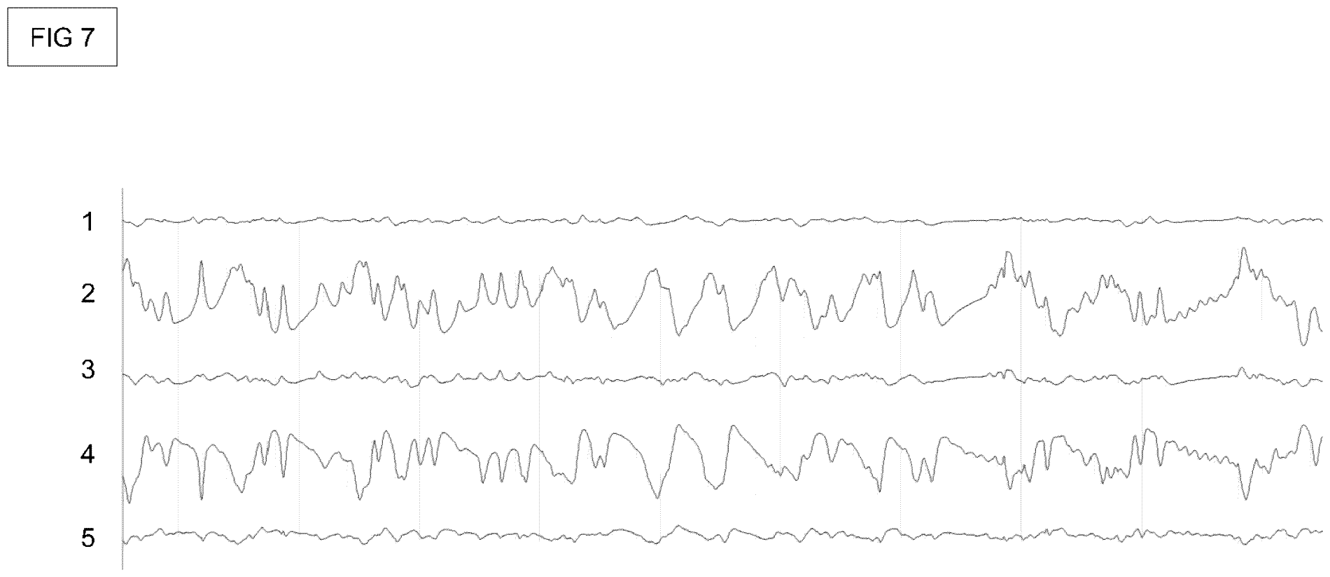

Data in FIG. 7 represents “synthetic” bipolar EEG tracings generated mathematically from adjacent contacts recording referential EEG data in the experiment outlined in FIG. 6. Time on the x-axis is measured in seconds per division. Channels labelled on the y-axis range from 1 (deepest pair) to 5(shallowest pair) within the brain.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

Please note that the following claims are provisional claims only, and are provided as examples of possible claims and are not intended to limit the scope of what may be claimed in any future patent applications based on the present application. Integers may be added to or omitted from the example claims at a later date so as to further define or re-define the invention.

1. An intracranial electroencephalographic (EEG) device comprising:
a ground electrode;
a reference electrode; and
a cortical recording array comprising at least one recording element,
wherein each of the ground electrode, the reference electrode and the cortical recording array are longitudinally arranged along a same axis of a single, cylindrically-shaped support structure, and
wherein when the EEG device is properly implanted in a subject's brain, the ground electrode and the reference electrode are positioned in a non-gray matter anatomic space and the cortical recording array is positioned to measure brain activity within a gray matter brain space located in a cerebral cortex.

2. The EEG device of claim 1, wherein the cortical recording array comprises between 1-10 recording elements.

3. The EEG device of claim 1, wherein the cortical recording array is positioned within or adjacent to the gray matter brain space of the cerebral cortex.

4. The EEG device of claim 1, wherein the EEG device further comprises physiological sensors capable of measuring intracranial pressure, oxygen concentration, glucose level, blood flow or tissue perfusion, tissue temperature, electrolyte concentration, tissue osmolarity, a parameter relevant to brain function and/or health, or any combination thereof.

5. The EEG device of claim 1, wherein the reference electrode and the ground electrode are positioned in different non-gray matter anatomic spaces.

6. The EEG device of claim 1, wherein when the non-gray matter anatomic space is selected from:
a subgaleal space;
a subcortical white matter space;
a space within a skull fixation device; or
a cerebral ventricle space.

7. The EEG device of claim 6, wherein:
the reference electrode is in the subgaleal space and the ground electrode is in the subcortical white matter space;
the ground electrode is in the subgaleal space and the reference electrode is in the subcortical white matter space;
the reference electrode is in the subgaleal space and the ground electrode is in the cerebral ventricle space;
the ground electrode is in the subgaleal space and the reference electrode is in the cerebral ventricle space;
the reference electrode is within a space of the skull fixation device and the ground electrode is in the subcortical white matter space;
the ground electrode is within a space of the skull fixation device and the reference electrode is in the subcortical white matter space;
the reference electrode is within a space of the skull fixation device and the ground electrode is in the cerebral ventricle space; or
the ground electrode is within a space of the skull fixation device and the reference electrode is in the cerebral ventricle space.

8. The EEG device of claim 6, wherein when the ground electrode is positioned in the subgaleal space, it is fixed to the single, cylindrically-shaped support structure at a distance between 1.5 cm and 10 cm distal to a most superficial recording element of the cortical recording array.

9. The EEG device of claim 1, wherein the EEG device further comprises a ventricular cerebrospinal fluid drainage function.

10. The EEG device of claim 1, wherein the ground electrode, the reference electrode, and/or the recording element is made of metal, an organic compound or other electrically conductive material.

11. The EEG device of claim 1, wherein the at least one recording element, the reference electrode, and the ground electrode are circumferentially arranged around the same axis of the single, cylindrically-shaped support structure.

12. The EEG device of claim 1, wherein the single, cylindrically-shaped support structure is made of plastic or a biocompatible material.

13. The EEG device of claim 1, wherein the single, cylindrically-shaped support structure is flexible or rigid.

14. The EEG device of claim 1, wherein the EEG device further comprises an interface connected to a processor capable of processing brain activity.

15. The EEG device of claim 14, wherein the interface is capable of filtering, amplifying, digitally transforming, compressing and/or transmitting brain activity detected by the cortical recording array.

16. The EEG device of claim 15, wherein the processor is capable of processing, filtering, amplifying, digitally transforming, comparing, storing, compressing, displaying, and/or otherwise transmitting the brain activity detected by the cortical recording array.

17. The EEG device of claim 14, wherein the processor comprises hardware and/or software that analyzes, manipulates, displays, correlates, stores and/or otherwise transmits brain electrical activity.

18. The EEG device of claim 17, wherein the processor identifies the ground electrode, the reference electrode and the cortical recording array in automated fashion for a selected electrode configuration, and wherein the processor uses the ground electrode selected in automated fashion to perform common-mode rejection for EEG signals recorded by a selected electrode configuration.

19. The EEG device of claim 1, wherein the ground electrode and the reference electrode are distal to the recording array.

20. The EEG device of claim 1, wherein the recording array is distal to the ground electrode and the reference electrode.
